(19) United States
(12) Patent Application Publication
(10) Pub. No.: US 2023/0302254 A1
(43) Pub. Date: Sep. 28, 2023

(54) EMBOLIZATION MICROCATHETER FOR DELIVERY OF BEADS TO PERIPHERAL VESSELS

(71) Applicant: ACCURATE MEDICAL THERAPEUTICS LTD., Rehovot (IL)

(72) Inventors: Tom DAGAN, Omer (IL); Yuval ZIPORY, Modiin (IL); Osnat HARBATER, Raanana (IL); Eran MILLER, Moshav Beit Elazari (IL)

(21) Appl. No.: 18/018,565

(22) PCT Filed: Jul. 27, 2021
(86) PCT No.: PCT/IL2021/050905

§ 371 Date: Jan. 29, 2023

(30) Foreign Application Priority Data
Jul. 30, 2020 (IL) 276418

(51) Int. Cl. A61M 25/00; A61M 25/01

(52) U.S. Cl. CPC A61M 25/0052; A61M 25/0108; A61B 2017/1205

(57) ABSTRACT

An embolization microcatheter for embolization of peripheral arteries including an elongated tubular member comprising a distal end having a length of 150-500 mm and an outer diameter of less than 0.8 mm, wherein the elongated tubular member comprises a multiplicity sections, each having a length of 5 mm -150 mm, wherein a wall of each of the multiplicity of sections comprises a braid, a polymer formed around the braid and an inner liner coating an inner surface thereof; wherein the polymer of the multiplicity sections differ; and a distal tip having a length of 1 mm - 3 mm and extending between a proximal end of a first radiopaque marker of the elongated tubular member and a distal end opening of the elongated tubular member; the wall of the distal tip is devoid of braid.

### TECHNICAL FIELD

The present disclosure generally relates to the field of microcatheters for embolization, specifically to embolization catheters suitable for delivery of embolization beads to peripheral blood vessels.

### BACKGROUND

Transarterial embolization therapy, tumor embolization, or transcatheter arterial embolization (TAE), involves administration of embolization material (which may include chemotherapeutics or/and radiotherapeutics) directly to a tumor (for example, liver tumors), via a microcatheter.

Embolization of tumors is typically performed utilizing microcatheters due to the requirement for selectively affecting the tumor while preventing, as much as possible, damage to healthy tissue. A major problem associated with embolization is “non-target embolization”, where the embolic material travels to blood vessels, other than those directly feeding the target tumor or tumor region, thus damaging healthy tissues, resulting in unpleasant and even hazardous outcomes.

During embolization, especially of peripheral vessels, the embolization catheter must be advanced through small and often tortuous vessels. Accessibility to these vessels is difficult, if not precluded, using large and/or stiff microcatheters. Moreover, blood vessels in the body tend to go into spasm when manipulated, causing an ineffective embolic material delivery, so flexible microsized catheters are an absolute necessity.

A major drawback of trans-catheter embolization is that the embolization material, which is typically invisible, can be refluxed and reach non-target tissue and cause damage to them. In addition, reflux of embolization material may negatively affect the delivery of the embolization material to the target tissue, and thus impair treatment effectiveness and its clinical outcome.

Microcatheters with filter sections for delivery of embolization beads, while preventing backflow of the beads, have been disclosed by the inventors of the present applications. However, there remains a need for microcatheters capable of delivering embolization beads to peripheral blood vessels, i.e. microcatheters with a sufficiently small outer diameter for unhindered entry into peripheral blood vessels yet which do not cause clogging or kinking of the microcatheter.

### SUMMARY OF THE INVENTION

The present disclosure relates to embolization microcatheters which are suitable for passing through and/or reaching peripheral arteries (typically 1.7 or 1.9 French (Fr)), while also facilitating unhindered delivery of embolization beads.

This is advantageously achieved by the unique structure of the wall of the embolization microcatheters which has an outer diameter of less than 0.8 mm, yet is still trackable and torque resistant. The large majority of the wall is made of a braid, a polymer formed around the braid and an inner liner coating an inner surface of the wall. The distal tip of the microcatheter (approximately the last 1 mm - 1.5 mm) includes a radiopaque marker for positioning. The distal tip is further characterized by its wall being devoid of the braid. This may, according to some embodiments, advantageously compensate for the increased wall thickness caused by the radiopaque marker.

The herein disclosed embolization microcatheter further includes a filter section with a plurality of openings configured for outflow of fluids while preventing outflow of the beads, thus providing concentrated delivery of the embolization beads through the distal end opening of the microcatheter, while ensuring minimal backflow. Advantageously, the size, shape and distribution of the opening enables smooth delivery of the beads, despite the small diameter of the microcatheter. According to some embodiments, the plurality of side openings is distributed on at least five circumferential rings spaced apart from each other by 100 microns - 200 microns.

According to some embodiments, the wall of the tubular member includes a multiplicity of sections, each having a length of 5 mm -150 mm and each formed of a different polymer. This may advantageously on the one hand provide pushability and on the other hand provide efficient maneuvering through tortuous blood vessels.

According to some embodiments, there is provided an embolization microcatheter for embolization of peripheral arteries comprising: an elongated tubular member comprising a distal end extending from a proximal marker to a distal end opening, the distal end having an outer diameter of about 0.7 mm or less, wherein the elongated tubular member comprises a multiplicity sections, each having a length of 5 mm-120 mm, wherein a wall of each of the multiplicity of sections comprises a braid, a polymer formed around the braid and an inner liner coating an inner surface thereof; wherein the polymer of at least some of the multiplicity sections differ and wherein a thickness of the wall is less than 100 microns; a distal tip having a length of 0.5 mm - 3 mm and extending between a proximal end of a distal radiopaque marker of the elongated tubular member and a distal end opening of the elongated tubular member; and wherein the wall of the distal tip is devoid of braid.

According to some embodiments, the inner lumen of the distal end is in a range of 0.3 mm- 0.7 mm. According to some embodiments, the inner lumen of the distal end is in a range of 0.35 mm - 0.55 mm. According to some embodiments, the inner lumen of the distal tip is below 0.5 mm.

According to some embodiments, the thickness of the wall is less than 90 microns.

According to some embodiments, the braid is made of tungsten wires. According to some embodiments, the braid is made of wires having a diameter of 15-20 microns. According to some embodiments, the braid has a picks per inch (PPI) of 150-220.

According to some embodiments, the multiplicity of sections comprises at least 5 sections. According to some embodiments, the multiplicity of sections comprises at least 9 sections. According to some embodiments, the distal most section of the multiplicity of sections has a length of 5-15 mm.

According to some embodiments, the embolization microcatheter further comprises a distal radiopaque marker, wherein the distal radiopaque marker comprises a metal marker band.

According to some embodiments, the distal most section of the multiplicity sections comprises a filter formed in the wall of the elongated tubular member, the filter comprising a plurality of side openings, the plurality of side opening distributed in at least 5 circumferential rings spaced apart from each other by 100 microns - 200 microns.

According to some embodiments, the plurality of openings is in the form of axial slits. According to some embodiments, the axial slits have a length of about 100-150 microns and a height of about 20-40 microns.

According to some embodiments, the distal most of the circumferential rings of the distal most filter section is positioned about 2-6 mm proximally to the distal end opening.

According to some embodiments, the plurality of side opening is distributed in at least 8 circumferential rings. According to some embodiments, each of the circumferential rings comprises 4-8 axial slits. According to some embodiments, the proximal most circumferential ring of the at least 5 circumferential rings comprises fewer side openings than rings distal thereto. According to some embodiments, the proximal most circumferential ring comprises 1-3 side openings. According to some embodiments, the side openings, of the proximal most circumferential section, is circumferentially shifted relative to the side openings in its neighboring circumferential section.

According to some embodiments, there is provided an elongated tubular member terminating with a distal end opening, the elongated tubular member comprising: a distal end extending 200-500 mm from the distal end opening toward a proximal end of the elongated tubular member, wherein an outer diameter of the elongated tubular member is less than 0.8 mm, wherein the elongated tubular member comprises a multiplicity sections, each having a length of 5 mm -150 mm, wherein a wall of each of the multiplicity of sections comprises a braid, a polymer formed around the braid and an inner liner coating an inner surface thereof; wherein the polymer of the multiplicity sections differ, wherein a thickness of the wall is less than 100 microns, and wherein the braid is made of wires having a diameter of 15-18 microns and has a picks per inch (PPI) of 200-350

According to some embodiments, the inner lumen of the distal end is in a range of 0.35 mm - 0.6 mm. According to some embodiments, the outer diameter of the distal end of the elongated tubular member is below 0.75 mm.

According to some embodiments, the thickness of the wall is less than 90 microns.

According to some embodiments, the braid is made of tungsten wires.

According to some embodiments, the multiplicity of sections comprises at least 5 sections. According to some embodiments, the multiplicity of sections comprises at least 9 sections. According to some embodiments, the distal most section of the multiplicity of sections has a length of 10-20 mm.

According to some embodiments, the embolization microcatheter further comprises a radiopaque marker comprises According to some embodiments, the radiopaque marker comprises a metal marker band.

According to some embodiments, the distal most section of the multiplicity sections comprises a filter formed in the wall of the elongated tubular member, the filter comprising a plurality of side openings, the plurality of side opening distributed in at least 5 circumferential rings spaced apart from each other by 100 microns - 200 microns.

According to some embodiments, the plurality of openings is in the form of axial slits. According to some embodiments, the axial slits have a length of about 100-150 microns and a height of about 20-40 microns.

According to some embodiments, the distal most of the circumferential ring of the distal most filter section is positioned about 2-6 mm proximally to the distal end opening.

According to some embodiments, the plurality of side opening is distributed in at least 8 circumferential rings. According to some embodiments, each of the circumferential rings comprises 4-8 axial slits.

According to some embodiments, the proximal most circumferential ring of the at least 5 circumferential rings comprises fewer side openings than rings distal thereto. According to some embodiments, the proximal most circumferential ring comprises 1-3 side openings. According to some embodiments, the side openings, of the proximal most circumferential section, is circumferentially shifted relative to the side openings in its neighboring circumferential section.

Certain embodiments of the present disclosure may include some, all, or none of the above characteristics. One or more technical advantages may be readily apparent to those skilled in the art from the figures, descriptions and claims included herein. Moreover, while specific characteristics have been enumerated above, various embodiments may include all, some or none of the enumerated characteristics.

In addition to the exemplary aspects and embodiments described above, further aspects and embodiments will be further expanded upon in the figures and the following detailed descriptions.

### BRIEF DESCRIPTION OF THE FIGURES

The features, nature and advantages of the present disclosure will become more apparent from the detailed description set forth below when taken in conjunction with the drawings in which like reference characters identify correspondingly throughout. Identical structures elements or parts that appear in more than one figure are generally labeled with the same number in all the figures in which they appear. Alternatively, elements or parts that appear in more than one figure may be labeled with different numbers in the different figures in which they appear. The dimensions of the components and features in the figures were chosen for convenience and clarity of presentation and are not necessarily shown to scale. The figures are listed below.

FIG. 1A schematically illustrates a microcatheter comprising an outer layer including a plurality of sections, the plurality of sections made of different polymeric materials, according to some embodiments.

FIG. 1B schematically illustrates a perspective, cutaway view of the distal end of the microcatheter of FIG. 1A illustrating the outer layer, the strike layer, the inner layer, the braided skeleton located between the inner layer and the outer layer.

FIG. 2A schematically illustrates an embolization microcatheter with a fluid barrier forming section, according to some embodiments.

FIG. 2B schematically illustrates a magnified and partially exposed view of the distal end of the microcatheter of FIG. 2A, according to some embodiments.

FIG. 2C schematically illustrates a magnified and partially exposed view of the distal tip of the microcatheter of FIG. 2A, according to some embodiments.

FIG. 2D schematically illustrates a slit formed by selective cutting through the wall of a fluid barrier forming section, such as the fluid barrier forming section of the embolization microcatheter of FIG. 2A, according to some embodiments.

FIG. 3 schematically illustrates an optional slit pattern for an embolization microcatheter such as the embolization microcatheter of FIG. 2A, according to some embodiments.

FIG. 4 schematically illustrates another optional slit pattern for an embolization microcatheter, such as the embolization microcatheter of FIG. 2A, according to some embodiments.

### DETAILED DESCRIPTION OF THE INVENTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will also be apparent to one skilled in the art that these concepts may be practiced without specific details being presented herein. In some instances, well-known features may be omitted or simplified in order to avoid obscuring the disclosure.

One of the main challenges of embolization microcatheters is ensuring a small enough outer diameter to facilitate entry into peripheral vessels, while also ensuring unhindered delivery of embolization beads (including unhindered flow within the lumen of the microcatheter) as well as a catheter wall which is strong, trackable and kink resistant.

Advantageously, these requirements are met by the herein disclosed microcatheter and the structural characteristics of its tubular wall.

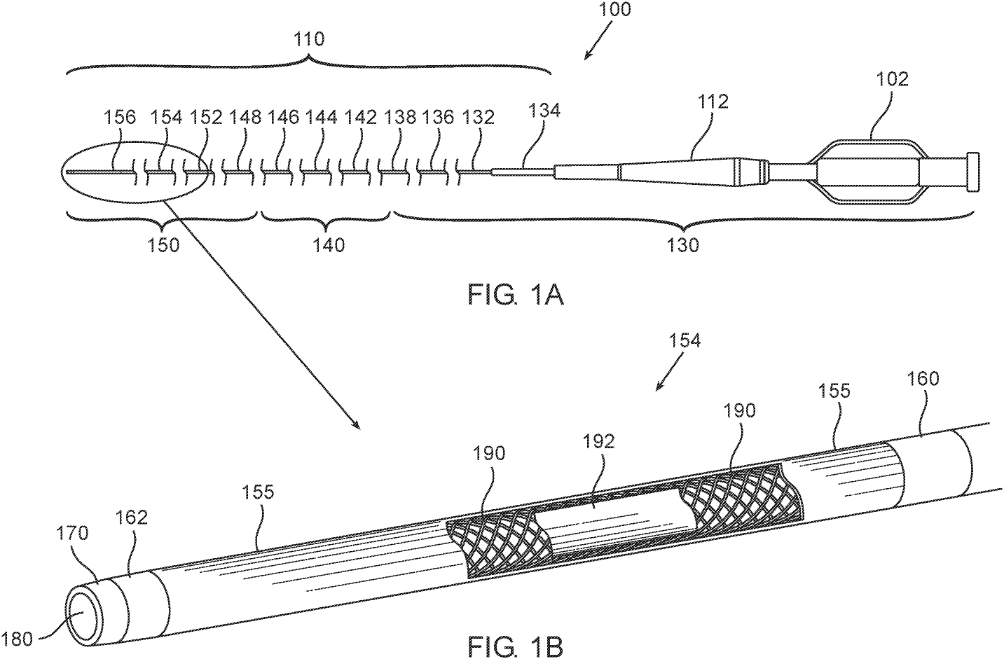

Reference is now made to FIG. 1A, and FIG. 1B, which schematically illustrate an embolization microcatheter 100 and magnified/exposed views of a distal part thereof.

As used herein, the terms “embolization”, “transcatheter embolization”, “transcatheter arterial embolization” and “TAE” may be used interchangeably and refer to the passage and lodging of an embolus within the bloodstream for therapeutic purposes, for example, as a hemostatic treatment of bleeding or as a treatment for some types of cancer by deliberately blocking blood vessels to starve the tumor cells.

Embolization microcatheter 100 includes an elongated tubular member 110. The proximal end 130 of microcatheter 100 includes a hub 102 which is molded on or otherwise attached to elongated tubular member 110 of microcatheter 100.

Hub 102 is configured to allow access to the lumen of elongated tubular member 110 for a variety of functions, such as the injection of fluids or drugs, or the introduction of guidewires. Hub 102 optionally includes a strain relief 112, preferably mechanically coupled to hub 102. Strain relief 112 may be made of a polymeric material and may, as illustrated, be tapered at its distal end. Strain relief 112 and be configured to provide structural support to elongated tubular member 110, to prevent it from kinking.

According to some embodiments, the wall of elongated tubular member 110 may include a plurality of sections, each section characterized by the polymers utilized. According to some embodiments, a plurality of sections may include 3, 4, 5, 6, 7, 8, 9, 10, 15, 20 or more sections. Each possibility is a separate embodiment.

According to some embodiments, the different polymeric layers may contribute to different characteristics of the layer/section and thus of elongated tubular member 110. For example, the different polymeric layers may contribute to the elasticity, flexibility, stretch-ability, strength, hardness, rigidity, ultimate tensile strength, elongation or any other characteristic of the layer and thus the microcatheter. Each possibility is a separate embodiment.

The proximal 130 end of elongated tubular member 110, attached to strain relief 112, includes first section 132. The outer layer of section 132 may be made of a polymeric material having a relatively high hardness such as a polyether block amide having a hardness of about 70 D shore and/or a flexural modulus of about 74,000 psi. According to some embodiments, proximal end 132 may have a length of 600-1300 mm (e.g. about 1000 mm).

Optionally, part of section 132 may include a heat shrink material 134 covering the joint between strain relief 112 and elongated tubular member 110.

Adjacent section 132, is a second section, section 136, which is slightly softer. The outer layer of section 132 may be made of polymeric material having a hardness of about 60D-70D shore and/or a flexural modulus between 41,000 psi-74,000 psi. Section 136 may have a length of 10-40 mm, or 20-30 mm e.g. 25 mm. Section 136 may be followed with a slightly softer section, section 138, which may be made of polymeric material having a hardness about 60-65D shore and/or a flexural modulus of about 41,000 psi. Section 138 may have a length of 60-80 mm, e.g. 70 mm.

According to some embodiments, the polymeric material of section 138 is softer than that of section 136. According to some embodiments, the polymeric material of section 136 is softer than that of section 132.

Intermediate part 140 of elongated tubular member 110 includes section 142 having an outer layer which may be made of a polyether block amide or other suitable polymer having a hardness of about 55 D shore and/or a flexural modulus of about 25,000 psi, section 144 having an outer layer made of a polymeric material having a hardness of about 55 D shore such as a polycarbonate-based thermoplastic urethane having a hardness of about 50 D shore, section 146 having an outer layer made of a one or more polycarbonate-based thermoplastic urethanes having a hardness between 55 D shore and 95 A shore, and section 148 having an outer layer made of a one or more polycarbonate-based thermoplastic urethanes having a hardness of 95 A shore. Section 142 may have a length of 50-90 mm, e.g. 70 mm. Section 144, may have a length of 80-1100 mm (e.g. about 90 mm). Section 146 may have a length of 50-70 mm (e.g. about 65 mm). Section 148 may have a length of 5-30 mm (e.g. about 15 mm).

According to some embodiments, the polymeric material of section 148 is softer than that of section 146. According to some embodiments, the polymeric material of section 146 is softer than that of section 144. According to some embodiments, the polymeric material of section 144 is softer than that of section 142.

Distal end 150 of elongated tubular member 110 includes sections 152 having an outer layer made of a polymeric material having a hardness of about 95 A shore, such as, for example, a polycarbonate-based thermoplastic urethane having a hardness of 95 A shore and a length of 10-40 mm (e.g. about 25 mm) and 154 having an outer layer made of a polymeric material having a hardness of about 85 A shore such as, for example, a polycarbonate-based thermoplastic urethane having a hardness of about 85 A and a length of about 3-10 mm (e.g. about 6 mm). The polymer of section 154 further includes a polymeric marker such as, but not limited to, tantalum powder and section 156 having an outer layer made of a polymeric material having a hardness of about 80 A shore such as a polycarbonate-based thermoplastic urethane having a hardness of about 85 A shore having a length of about 5-15 mm (e.g. about 9.5 mm).

According to some embodiments, elongated tubular member 110 may have an outer diameter in the range of 0.5 mm-1.5 mm or in the range of 0.55 mm - 1.0 mm. According to some embodiments the outer diameter of tubular member 110 may vary from its proximal end to its distal end. According to some embodiments, the outer diameter of tubular member 110 may be gradually decreasing from its proximal end to its distal end.

According to some embodiments, the outer diameter of the proximal end of tubular member 110, including section 132, may be in the range of 0.8-1.0 mm, such as, but not limited to, about 0.95 mm. According to some embodiments, the outer diameter of intermediate section of the tubular member 110 may be in the range of 0.7-0.9 mm. According to some embodiments, the outer diameter of the tubular member may decrease from about 0.9 mm to about 0.7 mm towards the distal end. According to some embodiments, the outer diameter of distal end of tubular member 110 may be in the range of 0.5-0.75 mm. According to some embodiments, the outer diameter of the tubular member may decrease from about 0.75 mm to about 0.55 mm towards the distal end opening. According to some embodiments, the outer diameter of distal tip 170 may be in the range of 0.55-0.65 mm. According to some embodiments, the outer diameter of distal tip 170 may be about 0.56 mm along the entire length thereof except over marker band 162 over which the outer diameter may be about 0.6-0.65 mm.

According to some embodiments, the outer diameter of section 156 is smaller than the outer diameter of section 154. According to some embodiments, the outer diameter of section 154 is smaller than the outer diameter of section 152. According to some embodiments, the outer diameter of section 152 is smaller than the outer diameter of section 148. According to some embodiments, the outer diameter of sections 148 and 146 is smaller than the outer diameter of section 144. According to some embodiments, the outer diameter of sections 144 is smaller than the outer diameter of section 142. According to some embodiments, the outer diameter of sections 142 is smaller than the outer diameter of section 136 and 132.

According to some embodiments, the outer diameter is essentially constant along a same section.

According to some embodiments, elongated tubular member 110 may have an inner diameter in the range of 0.35-0.65 or 0.4-0.60 mm. According to some embodiments, the inner diameter may be larger at the proximal end than at the distal end. According to some embodiments, the inner diameter of elongated tubular member 110 may be about 0.55 mm along the entire length of elongated tubular member 110 except the distal tip 170 (encompassing the last about 5-15 mm of the microcatheter, e.g. the last 10 mm), which may have an inner diameter of about 0.35-0.50 mm (e.g. about) 0.42 mm. According to some embodiments, the section extending from distal tip 170 to about 60-90 mm (e.g. about 70 mm) proximal thereto may be tapered.

As used herein, the term “distal end opening” refers to the end opening of the microcatheter leading into the lumen thereof. According to some embodiments, distal end opening 180 defines the termination of the microcatheter. According to some embodiments, distal end opening 180 may have an inner diameter essentially equal to the inner diameter of the microcatheter lumen. According to some embodiments, the distal end opening 180 may have an inner diameter which is smaller than the inner diameter of the microcatheter lumen leading to a narrowing of the lumen toward the end thereof.

Distal end 150 of elongated tubular member 110 may include a proximal marker 160 and a distal marker 162 (also seen in FIG. 1B). According to some embodiments, proximal marker 160 may be a radiopaque powder embedded the outer layers, as described herein above with regards to section 154. According to some embodiments, proximal marker 160 may be positioned approximately 5-20 or 10-15 mm from the distal end opening 180. According to some embodiments, distal marker 162 may be a radiopaque alloy submerged in outer layer of distal tip 170. According to some embodiments, distal marker 162 may be positioned approximately 0.25-1 mm proximally from distal end opening 180.

Reference is now made to FIG. 1B which schematically illustrates a perspective, cutaway view of the distal part of distal end 150 of microcatheter 100 shown in FIG. 1A extending from proximal marker 160 to distal end opening 180. As seen from the exploded view, underneath outer layer 155 is a braid 190.

According to some embodiments, braid 190 extends along the entire length of shaft elongated tubular member 110. Alternatively, braid 190 extends along the entire length of elongated tubular member 110, apart from distal tip 170. According to some embodiments, braid 190 may extend from the proximal end of tubular member 110 until distal marker 162. According to some embodiments, the part of tubular member 110 extending from distal marker 162 to distal end opening 180 may be devoid of braid.

Preferably, braid 190 has a picks per inch (PPI) ensuring that, in combination with a low durometer polymer, a flexible distal end is obtained, and in combination with a polymer having a higher durometer a relatively stiff proximal end is provided.

According to some embodiments, braid 190 may be made of a plurality of wires.

As used herein the terms “braid” and “braided skeleton” may refer to a structural element, such as a tubal element formed of a plurality of interlaced wires. According to some embodiments, the braid may be formed of at least three interlaced wires forming a tube. According to some embodiments, the braid may include 8-48 wires or 12-32 wires. Each possibility is a separate embodiment. As a non-limiting example, the braid may include 16 wires.

According to some embodiments, the wires forming the braid may have a diameter in the range of 10-40 microns or 12-20 microns or 15-18 microns or any other suitable diameter within the range of 10-60 microns. Each possibility is a separate embodiment. As a non-limiting example, the wires forming the braid may have a diameter of 18 microns.

According to some embodiments, the braid may be made from tungsten, stainless steel, Nickel titanium (also referred to as Nitinol), nitinol, cobalt chrome, platinum iridium, nylon or any combination thereof. Each possibility is a separate embodiment. As a non-limiting example, the wires may be tungsten wires.

According to some embodiments, at least some of the wires forming the braided skeleton may be braided in a same or opposite direction, i.e. left/right handed. Advantageously, the braiding structure allows good torque-ability (better than a coiled skeleton), low flexural rigidity (i.e. good flexibility), good push-ability (better than a coiled skeleton), and superior kink-resistance.

According to some embodiments, at least some of the wires forming the braided skeleton may be non-circular/round.

According to some embodiments, the braided skeleton may have a wire arrangement of 100-400 picks per inch (PPI), 150-375 PPI or 200-350 PPI. Each possibility is a separate embodiment. As a non-limiting example, the braided skeleton may have a wire arrangement of about 250 PPI. As another non-limiting example, the braided skeleton may have a wire arrangement of about 275 PPI. As another non-limiting example, the braided skeleton may have a wire arrangement of about 300 PPI. As another non-limiting example, the braided skeleton may have a wire arrangement of about 325 PPI. As another non-limiting example, the braided skeleton may have a wire arrangement of about 350 PPI. Those skilled in the art will appreciate the term picks per inch (PPI) is a measurement of braid wire density and represents the number of picks (e.g. weft wires) per inch of braid.

According to some embodiments, the PPI of the braid may differ along the length of tubular member 110. According to some embodiments, the PPI at the distal tip is higher than at sections distal thereto. As a nonlimiting example, the PPI of braid 190 at the distal tip may be about 200 PPI whereas the PPI of sections proximal thereto may be about 160 PPI. According to some embodiments, braid 190 includes a transition zone at which the PPI of the braid transition is from 200 PPI to 160 PPI.

Underneath braid 190 is an inner liner 192, which may be made of Polytetrafluoroethylene (PTFE). According to some embodiments, inner liner 192 may have a thickness of about 5-25 microns or 5-15 microns. Each possibility is a separate embodiment. According to some embodiments, the liner may be ram-extruded liner.

According to some embodiments, the total thickness of the wall of the distal end of the elongated tubular member does not exceed about 100 microns. According to some embodiments, the total thickness of the wall of the distal end of the elongated tubular member does not exceed about 90 microns. According to some embodiments, the total thickness of the wall of the distal end of the elongated tubular member does not exceed about 80 microns.

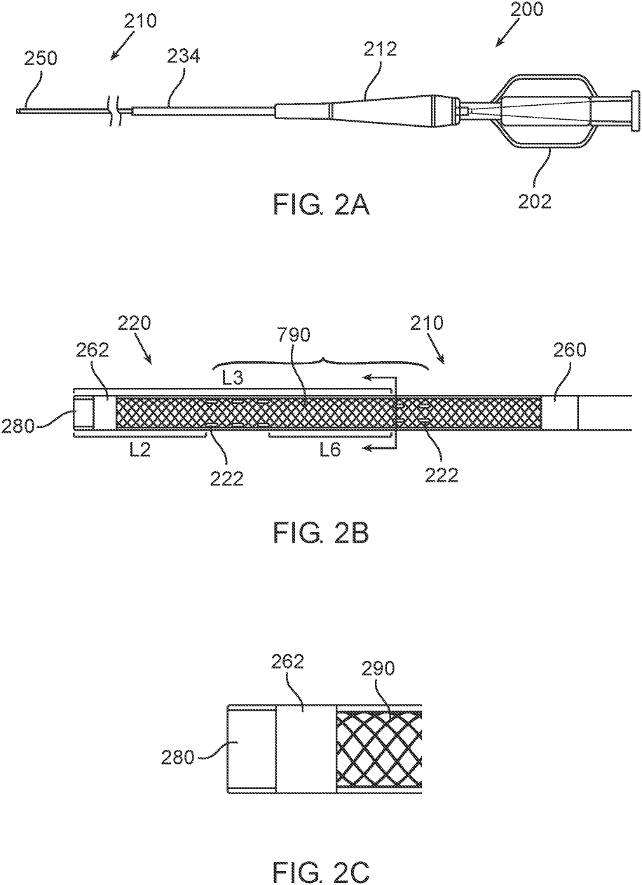

Reference is now made to FIG. 2A-FIG. 2D, which schematically illustrate an embolization microcatheter 200 and magnified/exposed views of parts thereof. Embolization microcatheter 200 may be similar to embolization microcatheter 100 apart from embolization microcatheter 200 also including a filter 220.

The proximal end of microcatheter 200 includes a hub 202 which is molded on or otherwise attached to microcatheter 200. Hub 202 is configured to allow access to the lumen of microcatheter 100 for a variety of functions, such as the injection of fluids or drugs, or the introduction of guidewires. Hub 202 includes a strain relief 212, preferably mechanically coupled to hub 202. Strain relief 212 may be made of a polymeric material and may, as illustrated, be tapered at its distal end. Strain relief 212 is configured to provide structural support to microcatheter 200, thereby preventing/minimizing kinking of microcatheter 200.

Reference is now made to FIG. 2B which schematically illustrates a partially exposed view of distal end 250 of microcatheter 200 shown (the portion of the distal end 250 extending between proximal marker 260 and distal marker 262 being exposed). Similarly to elongated tubular member 110, underneath the outer layer is a braid 290, which is essentially the same as braid 190.

Filter 220 including with a plurality of penetrating side openings formed in the wall of elongated tubular member 210, schematically illustrated in FIG. 2B.

As used herein, the term “plurality” with referral to the side openings refer to 2 or more, 3 or more, 5 or more, 10 or more, 15 or more, 20 or more or 25 or more axial slits. Each possibility is a separate embodiment

According to some embodiments, the filter 220 may be an integral part of elongated tubular member 110 and may extend along a length of 0.3 mm-20 mm, such as 1 mm-10 mm, 1 mm-5 mm, 1.5 mm-5 mm, 2 mm-5 mm or any other in-between suitable length. Each possibility is a separate embodiment.

According to some embodiments, filter 220 may have a total open area, formed by the side openings, in the range of 0.2-1 mm², 0.2-0.6 mm², 0.3-1 mm2, 0.3-0.5 mm², 0.4-0.6 mm², 0.5-1.5 mm², 1.0-3.5 mm², 1.5-4 mm², 2.0-3.5 mm² or any other suitable area within the range of 0.1-4 mm². Each possibility is a separate embodiment. According to some embodiments, at least 5%, at least 10%, at least 15% of filter 220 is open area formed by the side openings. According to some embodiments, 5%-30%, at least 7%-25%, 7%-20%, 5%-15% of filter 220 is open area formed by the side openings. Each possibility is a separate embodiment.

According to some embodiments, side openings 225 may be formed by selective cutting (e.g. selective laser cutting), that is, without cutting the wires forming braid 290 as illustrated in FIG. 2D. According to some embodiments, the part of the liner positioned below the wires remains intact. According to some embodiments, both the polymeric layer and the inner liner positioned between the wires of braid 290 are penetrated when forming the slits. Advantageously, the selective cutting of the polymeric layer (leaving braid 290 essentially intact may provide subdivision of at least some of the side-openings into two or more sub-side-openings (illustratively depicted as side opening 225a and 292b) separated by the braid but not by the polymeric outer layer.

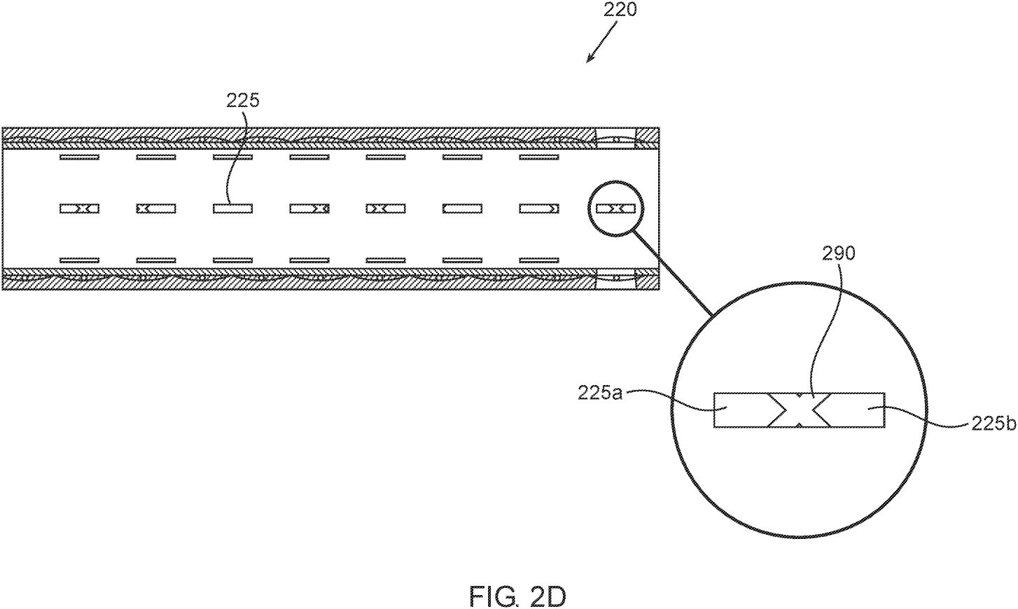

One optional structure of filter 220 is provided in FIG. 3. According to some embodiments, the structure is suitable for a 1.9 Fr embolization microcatheter. As seen in FIG. 3, filter 220 may include three filter sections, each filter section comprising a plurality of side openings 225, distributed in circumferential rings around elongated filter 220.

According to some embodiments, filter section 1 may include 1-10 or 2-8 or 4-7 rings (here illustrated as 7 rings) of side openings. According to some embodiments, each of the rings may include 1-8 side openings or 2-6 side openings, such as, but not limited to 6 side openings per ring. According to some embodiments, filter section 221 may include a total of 20-50 or 25-60 side openings, such as but not limited to 42 side openings. According to some embodiments, the distal most of the rings of filter section 1 may be positioned about 3-10 mm or 4-8 mm, such as but not limited to about 5 mm from the distal end opening 180.

According to some embodiments, filter section 2 may include 1-5 or 2-4 rings of side openings, such as, but not limited to 3 rings of side opening. According to some embodiments, each of the rings may include 1-6 side openings or 2-4 side openings, such as, but not limited to 4 side openings per ring. According to some embodiments, filter section 2 may include a total of 5-20, 6-16 side openings, such as but not limited to 12 side openings.

According to some embodiments, side openings of filter section 2 may be circumferentially shifted relative to side openings of filter section 1.

According to some embodiments, filter section 3 may include 1-5 or 2-4 rings of side openings, such as, but not limited to 2 rings of side opening. According to some embodiments, each of the rings may include 1-4 side openings or 1-3 side openings per ring, such as but not limited to 2 side openings per ring. According to some embodiments, filter section 3 may include a total of 2-6, or 2-4 side openings, such as but not limited to 4 side openings.

According to some embodiments, the side openings of a first ring in section 3 may be circumferentially shifted relative to side openings of a second ring in section 3. According to some embodiments, side openings of filter section 3 may be circumferentially shifted relative to side openings of filter section 1.

According to some embodiments, side openings 225 may have a dimension of about 150×25 microns, about 150×30 microns, about 125×30 microns or about 100×30 microns.

According to some embodiments, each ring of side openings of filter sections 1-3 may be spaced apart from its neighboring ring by 100-200 microns or by 120-180 microns, such as but not limited to 150 microns.

Advantageously, filter 220 may be formed by selective cutting of the polymeric layer (leaving braid 290 essentially intact). According to some embodiments, at least some of side openings 225 may include sub-side-openings (such as sub-side-opening 225a and 225b in FIG. 2D) separated by braid 290, but not by the polymeric outer layer.

According to some embodiments, the slits may be positioned at a same or a different longitudinal position. Each possibility is a separate embodiment. According to some embodiments the distribution of the slits may be staggered, zig-zagged or any other suitable even or uneven distribution.

Advantageously, the filter 220 may be configured for kink-free bending despite the plurality of side openings formed in the wall thereof. According to some embodiments, the flexibility of the filter 220 is determined by the number of side openings, their minimal cross-sectional dimension, their width, length spacing, geometry, distance from distal outlet etc., as essentially described herein, may enable kink-free bending thereof.

As used herein the term “kink-free bending” may refer to a bending of filter 220, which does impede flow therethrough. According to some embodiments, filter 220 may be configured for kink-free bending at an angle of about 180 degrees. According to some embodiments, filter 220 may be configured for kink-free bending at a minimum bending radius in the range of about 0.5 to 1.5 mm, for example 0.5 to 1.2, 0.5 to 1 mm, or any radius in-between.

Advantageously, microcatheter 200 including filter 220 provides effective reflux prevention, which requires a relatively high density of side openings, while a small kink-free radius (e.g. in the range of 0.5 to 1.5 mm) and tensile strength of at least 5N is still ensured.

According to some embodiments, the microcatheter 200 may have a length of at least 50 cm, at least 60 cm, at least 75 cm, or at least 1 m. Each possibility is a separate embodiment. Each possibility is a separate embodiment.

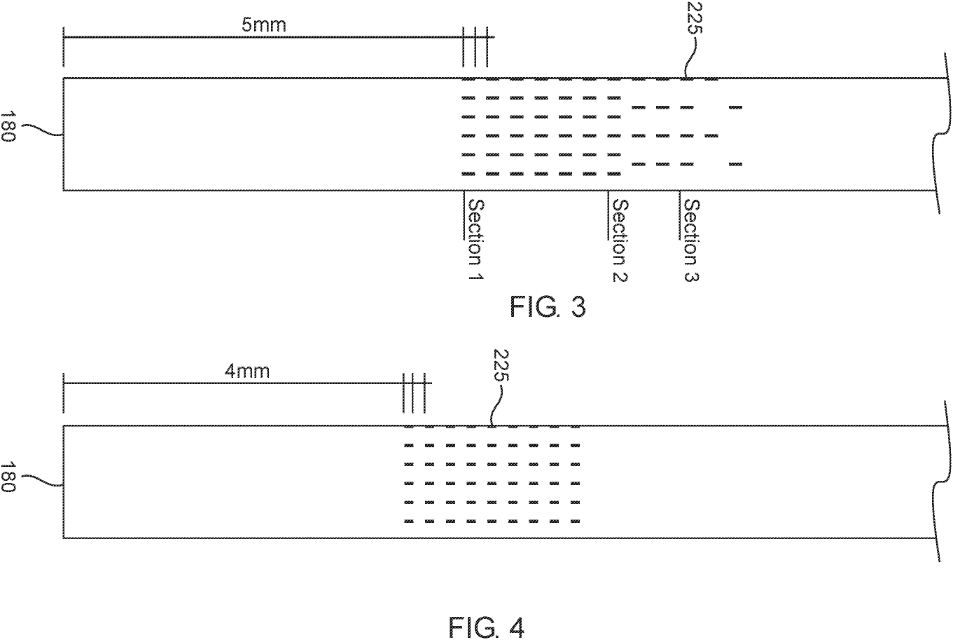

Another optional structure of filter 220 is provided in FIG. 4. According to some embodiments, the structure is suitable for a 1.7 Fr embolization microcatheter. As seen in FIG. 4, filter 220 may include a plurality of side openings 225, distributed in circumferential rings around elongated filter 220.

According to some embodiments, filter 220 may include 2-20 or 5-15 or 6-10 rings (e.g. 9 rings) of side openings. According to some embodiments, each of the rings may include 2-10 side openings or 4-8 side openings, such as, but not limited to 6 side openings per ring. According to some embodiments, filter 220 may include a total of 30-80 or 40-60 side openings, such as but not limited to 54 side openings. According to some embodiments, the distal most of the rings of filter section 1 may be positioned about 2-10 mm or 3-6 mm, such as but not limited to about 4 mm from the distal end opening 180.

According to some embodiments, side openings 225 may have a dimension of about 150x25 microns, about 150x30 microns, about 125x30 microns or about 100x30 microns.

According to some embodiments, each ring of side openings may be spaced apart from its neighboring ring by 500-200 microns or by 120-180 microns, such as but not limited to 150 microns.

Advantageously, filter 220 may be formed by selective cutting of the polymeric layer (leaving braid 290 essentially intact). According to some embodiments, at least some of side openings 225 may include sub-side-openings (such as sib-side-opening 225a and 225b in FIG. 2D) separated by braid 290, but not by the polymeric outer layer.

According to some embodiments, the slits may be positioned at a same or a different longitudinal position. Each possibility is a separate embodiment. According to some embodiments the distribution of the slits may be staggered, zig-zagged or any other suitable even or uneven distribution.

Advantageously, the filter 220 may be configured for kink-free bending despite the plurality of side openings formed in the wall thereof. According to some embodiments, the flexibility of the filter 220 is determined by the number of side openings, their minimal cross-sectional dimension, their width, length spacing, geometry, distance from distal outlet etc., as essentially described herein, may enable kink-free bending thereof.

As used herein the term “kink-free bending” may refer to a bending of filter 220, which does impede flow therethrough. According to some embodiments, filter 220 may be configured for kink-free bending at an angle of about 180 degrees. According to some embodiments, filter 220 may be configured for kink-free bending at a minimum bending radius in the range of about 0.5 to 1.5 mm, for example 0.5 to 1.2, 0.5 to 1 mm, or any radius in-between.

Advantageously, microcatheter 200 including filter 220 provides effective reflux prevention, which requires a relatively high density of side openings, while a small kink-free radius (e.g. in the range of 0.5 to 1.5 mm) and tensile strength of at least 5N is still ensured.

According to some embodiments, the microcatheter 200 may have a length of at least 50 cm, at least 60 cm, at least 75 cm, or at least 1 m. Each possibility is a separate embodiment. Each possibility is a separate embodiment.

As used herein, the terms “approximately” and “about” refer to +/-10%, or +/-5%, or +-2% vis-à-vis the range to which it refers. Each possibility is a separate embodiment.

While a number of exemplifying aspects and embodiments have been discussed above, those of skill in the art will envisage certain modifications, additions and subcombinations thereof. It is therefore intended that the following appended claims and claims hereafter introduced be interpreted to include all such modifications, additions and sub-combinations as are within their true spirit and scope.

1. An embolization microcatheter for embolization of peripheral arteries comprising:
an elongated tubular member comprising a distal end extending from a proximal marker to a distal end opening, the distal end having an outer diameter of about 0.7 mm or less,
wherein the elongated tubular member comprises a multiplicity of sections, each having a length of 5 mm -120 mm, wherein a wall of each of the multiplicity of sections comprises a braid, a polymer formed around the braid and an inner liner coating an inner surface thereof; wherein the polymer of at least some of the multiplicity sections differ and wherein a thickness of the wall is less than 100 microns;
a distal tip having a length of 0.5 mm - 3 mm and extending between a proximal end of a distal radiopaque marker of the elongated tubular member and a distal end opening of the elongated tubular member; and wherein the wall of the distal tip is devoid of braid.

2. The embolization microcatheter according to claim 1, wherein an inner lumen of the distal end is in a range of 0.3 mm - 0.7 mm.

3. (canceled)

4. The embolization microcatheter according to claim 1, wherein an inner lumen of the distal tip is below 0.5 mm.

5. The embolization microcatheter according to claim 1, wherein the thickness of the is less than 90 microns.

6. The embolization microcatheter according to claim 1, wherein the braid is made of tungsten wires.

7. The embolization microcatheter according to claim 1, wherein the braid is made of wires having a diameter of 15-20 microns.

8. The embolization microcatheter according to claim 1, wherein the braid has a picks per inch (PPI) of 150-220.

9. The embolization microcatheter according to claim 1, wherein the multiplicity of sections comprises at least 5 sections.

10. The embolization microcatheter according to claim 9, wherein the multiplicity of sections comprises at least 9 sections.

11. The embolization microcatheter according to claim 1, wherein the distal most section of the multiplicity of sections has a length of 5-15 mm.

12. The embolization microcatheter according to claim 1, wherein the distal radiopaque marker comprises a metal marker band.

13. The embolization microcatheter of claim 1, wherein the distal most section of the multiplicity sections comprises a filter formed in the wall of the elongated tubular member, the filter comprising a plurality of side openings, the plurality of side opening distributed in at least 5 circumferential rings spaced apart from each other by 100 microns - 200 microns.

14. The embolization microcatheter according to claim 13, wherein the plurality of openings is in the form of axial slits.

15. The embolization microcatheter according to claim 14, wherein the axial slits have a length of about 100-150 microns and a height of about 20-40 microns.

16. The embolization microcatheter according to claim 13, wherein a distal most of the circumferential rings of the distal most filter section is positioned about 2-6 mm proximally to the distal end opening.

17. The embolization microcatheter according to claim 13, wherein the plurality of side opening is distributed in at least 8 circumferential rings.

18. The embolization microcatheter according to claim 13, wherein each of the circumferential rings comprises 4-8 axial slits.

19. The embolization microcatheter according to claim 13, wherein a proximal most circumferential ring of the at least 5 circumferential rings comprises fewer side openings than rings distal thereto.

20. (canceled)

21. The embolization microcatheter according to claim 19, wherein the side openings, of the proximal most circumferential section, is circumferentially shifted relative to the side openings in its neighboring circumferential section.

22-39. (canceled)
